(19) United States
(12) Patent Application Publication
(10) Pub. No.: US 2023/0232873 A1
(43) Pub. Date: Jul. 27, 2023

(54) Dye for Foods, Nutritional Supplements, Cosmetic or Pharmaceutical Products

(71) Applicant: Döhler GmbH, Darmstadt (DE)

(72) Inventors: Roland BECK, Darmstadt (DE); Alexander LEICHT, Bickenbach (DE)

(73) Assignee: Döhler GmbH, Darmstadt (DE)

(21) Appl. No.: 18/007,995

(22) PCT Filed: Jun. 8, 2021
(86) PCT No.: PCT/EP2021/065208

§ 371 Date: Dec. 2, 2022

(30) Foreign Application Priority Data
Jun. 10, 2020 (DE) 10 2020 003 477.2

(51) Int. Cl. A23L 5/42; A23L 33/00; A61K 47/02; A23G 3/36; A23G 1/30; A23G 1/32; A23L 2/58

(52) U.S. Cl. CPC A23L 5/42; A23L 33/30; A61K 47/02; A23G 3/362; A23G 1/305; A23G 1/325; A23L 2/58

(57) ABSTRACT

For coloring foods, nutritional supplements, cosmetic or pharmaceutical products, the invention provides a dye, which contains at least one pigment in the form of a water-insoluble sulfate, carbonate, or phosphate of at least one alkaline earth metal, which is selected from the group consisting of calcium sulfate, magnesium phosphate, calcium phosphate, and magnesium carbonate.

### TECHNICAL FIELD

The invention relates to a dye for coloring foods, nutritional supplements, cosmetic or pharmaceutical products, as well as to the use of in particular sulfates and/or carbonates of the alkaline earth metals as pigment for coloring foods, nutritional supplements, cosmetic or pharmaceutical products, and a product of the food, pharmaceutical, or cosmetics industry, which contains such a dye.

### BACKGROUND

Pigments are dyes, coloring substances, which, in contrast to colorants, consist of particles, and which are virtually insoluble in the application medium. The application medium is thereby the substance, in which the pigment is incorporated, for example in binding agents, such as oils or plastics, or also in foods. Pigments can be differentiated according to their chemical structure, according to their optical properties, and according to their technical properties. The color stimulus of the pigments is created by means of absorption and remission of certain frequency portions of the visible light. What is decisive for the properties of the pigments are solid-state properties, such as crystal structure, crystal modification, particle size, and particle size distribution, the latter due to the specific surface.

For example, achromatic pigments (black and white pigments) and colored pigments belong to the inorganic pigments. When light beams impinge on a pigment or an object colored therewith, they can be scattered. The all-around deflection of the light beams is thereby referred to as “scattering”, whereby a differentiation is made between light refraction and light reflection. During the reflection, the light beam does not enter into the pigment, but is reflected back at the interface. During the light refraction, the light beam enters into the pigment and is deflected there. When impinging on a pigment or an object colored with it, respectively, light can additionally be absorbed by it. In most cases, several of these events collectively result in the observable color impression.

The optical properties of the white pigments result from a strong, non-selective light scattering, associated with a very low light absorption. What is decisive thereby for the color impression of the white pigments is a strong reflection. The latter takes place when the refractive indices between pigment and application medium differ greatly. In connection with the use in the surface treatment, the refractive index (n_{D}) is mainly a measure of the covering power. The covering power is greater, the greater the difference of the refractive indices between application medium and pigment. However, the refractive index depends on the spatial direction of the crystal, which is an unchangeable material property, which is why, for example, several values for different pigments depending on the water of crystallization can be found in the literature.

To assess the degree of whiteness, the refractive index alone is not very meaningful, however, because in particular the particle size and shape of the white pigments decisively influence the covering power and the shade. For example, white minerals with a lower refractive index, such as, e.g. chalk (n_{D}=1.55), can by all means attain a covering power, which is comparable to white pigments, such as titanium dioxide, because the particle size of a pigment in a given dispersion has a direct impact on the scattered light intensity. This scattering capacity, in turn, determines the optical properties of the finished product, such as, for example, the opacity.

The opacity or covering power of a given pigment dispersion is a measure for its ability to completely mask the substrate located therebelow. A pigment with a fine particle size is thus able to create an equivalent opacity in the case of lower concentrations, which is usually observed in the case of much higher concentrations when using a coarser material. The finer the particles, the better the covering capacity. However, the covering capacity drops again at a certain fineness of the pigment.

Achromatic inorganic pigments, which are used in particular to create optical whiteness in coating materials or as filler material in, e.g., plastics, are referred to as white pigments. The main fields of use of the white pigments are the paper, paint, plastics, and coatings industry, but they are also used in the food industry.

As the most well-known white pigment, titanium dioxide has a refractive index, which is significantly greater than that of most organic substances, which are used to bind colors (binding agents) (n_{D} approx. 2.6-2.7). This means that pigments of titanium dioxide scatter the light effectively, so that a well-covering white color results. The optimal size of the TiO₂ pigments thereby lies in the range of from 200 nm to 300 nm. In the food industry, it is used in high purity as food additive under the name E 171, for example in toothpaste, chewing gum, coated tables and coatings, as well as under CI 77891 in cosmetics.

In contrast to the soluble colorants, the pigment titanium dioxide can only be distributed very finely in a medium. Due to the fact that it is chemically stable, the chemical composition does not change under light, heat, and the influence of acid. Titanium dioxide is chemically stable, is considered to be inert, and is classified as non-toxic because it cannot be metabolized in the body. However, titanium dioxide can sometimes be stored in human tissue and can cause inflammation. It is furthermore suspected that the food coloring E171 contains a certain portion of nano material, for which studies proved an effect, which is hazardous to health. In June of 2017, the Committee for Risk Assessment of the European Chemicals Agency classified this substance to potentially be carcinogenic when absorbed by inhalation. Due to these health concerns, France has banned the use of titanium dioxide in foods since the beginning of 2020.

As alternative for the white pigmentation with titanium dioxide, calcium carbonate is known for coloring, colloquially referred to as lime or chalk, which, as white pigment, is likewise an approved food coloring (E 170). Calcium carbonate is generally approved for foods without maximum quantity restrictions and is used, e.g., in coated tables, coatings for foods, cheese, chewing gum, and baking agents. Even though this pigment is insoluble in water and grease, lightfast and heat-resistant, it is sensitive to acids, so that it thus dissolves in almost all food uses. Only very white and pure calcium carbonate can be used as white pigment. It can only be found in few parts of the world—calcium carbonate for the food industry is obtained from France, Turkey, and the USA. Compared to titanium dioxide, calcium carbonate has to additionally be used in significantly higher concentrations in order to attain an identical degree of whiteness.

The German patent application DE 10 2017 127 902 A1 describes the use of mixtures of calcium carbonate with phosphate particles, whereby phosphate particles are understood to be soluble as well as insoluble phosphates, which are to have a brightening effect in foods, such as caramels, jelly candies, salad dressings, sauces, beverages, etc. A brightening effect of calcium carbonate, however, is destroyed by the acids, which are present in the foods. The PKA value of carbon dioxide is 6.5, that is, foods with a pH value of below 6.5 dissolve the pigmentary calcium carbonate into soluble calcium salts, which then no longer have a whitening power. The soluble phosphates then likewise do not have any brightening effect whatsoever because they dissolve as well.

In addition to such calcium carbonate-containing mixtures, different starches and starch compositions—as described, for example, in WO2014074909—consisting of starch and dextrin and optionally further additives, are used as further substitutes for titanium dioxide in the food sector. The disadvantage of the use of starches is that they can be used only in dry uses without any further additives because they gelatinize otherwise and lose their whitening power. Starches are likewise not temperature stable and can thus not be used in uses, such as hard caramels, for example.

### SUMMARY

There is still a need for other white pigments, which can be used safely or without E numbers, respectively, in the food, cosmetics, or pharmaceutical industry. It was the object of the present invention to provide such a pigment. It was of particular interest to find a white pigment, which can be used to color foods in the confectionery sector or beverages.

The object is solved by means of a pigment for whitening foods, nutritional supplements, cosmetic or pharmaceutical products (such as hygiene products, health care products or medicines), which contains a sulfate, which is water-insoluble in particular at 200 and pH 7, carbonate, or phosphate of at least one alkaline earth metal. A substance, which is at most considered to be slightly soluble in water and of which preferably maximally a quantity of approx. 2.5 g·L⁻¹ is water-soluble at 20° C. and pH 7, is referred to as “water-insoluble”.

The invention provides a dye for coloring foods, nutritional supplements, cosmetic or pharmaceutical products, which contains at least one pigment in the form of a water-insoluble sulfate, carbonate, or phosphate of at least one alkaline earth metal, which is selected from the group consisting of calcium sulfate, magnesium phosphate, calcium phosphate, and magnesium carbonate.

The term “dye” is used for substances or mixtures, which can be added to an application medium or can be applied to such an application medium, in order to set the color of the application medium. The dye can comprise one pigment or several pigments. The dye can consist of one or several pigments or can be a composition, which contains one or several pigments. In addition to one or several pigments, the dye can additionally also contain at least one colorant

In contrast to “pigment”, the term “colorant” is used for substances, which are soluble in the application medium. A “dye” according to the invention can also be referred to below as “color”. The person of skill in the art in each case recognizes from the context, whether the color is present in powder form as solid or solid dispersion of the at least one pigment, or as suspension of the at least one pigment in a liquid. It can additionally be seen whether the respective color is white or a color from the spectrum of visible light.

For coloring cosmetic and/or pharmaceutical products, the dye contains at least one pigment in the form of a water-insoluble sulfate, carbonate, or phosphate of at least one alkaline earth metal, which is selected from the group consisting of calcium sulfate, magnesium phosphate, calcium phosphate, and magnesium carbonate.

In particular tri- and/or di-alkaline earth phosphates are possible as phosphates for the dye according to the invention because the dihydrogen phosphates—also referred to as “monophosphates”—usually have a water-solubility, which is too high for the use in foods, nutritional supplements, cosmetic or pharmaceutical products.

Magnesium or calcium can be used as alkaline earth metal, whereby calcium carbonate is excluded. Calcium sulfate, magnesium sulfate and/or magnesium carbonate are preferably used, calcium sulfate is particularly preferred, calcium sulfate anhydrite is most preferred. In a particularly preferred embodiment of the invention, it is provided that the dye contains only calcium sulfate, preferably anhydrite, as pigment, and particularly preferably consists of anhydrite. Anhydrite, in particular anhydrite III, anhydrite IIₛ, IIᵤ and/or anhydrite I can be used thereby as calcium sulfate.

Calcium sulfate (CaSO₄) is known in the food industry and is used there as firming agent, acidity regulator, and carrier, or is used as coagulant in the production of tofu. In nature, calcium sulfate occurs naturally as gypsum rock and mostly contains water of crystallization (CaSO₄·2H₂O—calcium sulfate dihydrate), but also pure CaSO₄ without water of crystallization, so-called anhydrite, occurs naturally. The production of calcium sulfate anhydrite by means of calcination (dry heating) of naturally derived calcium sulfate dihydrate does not change the color or composition of the substance. Calcium sulfate is pH- and temperature-stable and can be used as mineral source by the body, it thereby has a unique feature because it can be used both as calcium and sulfate source.

In a preferred embodiment of the invention, the pigment serves to whiten and/or color foods, in particular confectionery, coatings of confectionery, or beverages. Goods on the basis of hard caramel, gelatin-based or pectin-based fruit gummies, jelly or hard- and soft-coated tablets, e.g., can be considered to be confectionery.

In addition to one or several pigments, in particular a sulfate or a carbonate of an alkaline earth metal, the dye according to the invention can contain further common additives. Additives of this type can in particular be selected from the group comprising water, acids or alkalis, respectively, for setting the pH value, sweeteners, in particular sugar, isomalt and maltitol, salt, aromatic substances and/or flavorings, stabilizers, emulsifiers and thickening agents, for example gum arabic, as well as binding agents, in particular vegetable oils or polyols, gelatin, starch, and pectin.

In a further advantageous embodiment of the invention, it is provided that except for the at least one pigment, the dye contains at least one soluble colorant, in particular at least one colorant, which is soluble in the application medium. If a certain percentage of the pigment according to the invention is mixed to soluble colors, a higher brilliancy is attained, which is used to color surfaces, for example chocolate drops.

To attain an efficiency (covering power/opacity) of the pigment or dye according to the invention, respectively, which is approximately identical to the TiO₂ efficiency, for whitening in a corresponding food, cosmetic, or pharmaceutical product, it would have been expected that extremely higher required quantities of the pigment according to the invention, which preferably contains calcium sulfate, would have to be used. Due to the fact that the refractive index difference between the pigment according to the invention (e.g. a value of n_{D} 1.57 for CaSO₄ according to https://www.chemicalbook.com/ChemicalProductProperty_EN_cb5110067.htm) and the matrix (e.g. n_{D} 1.53 for hard caramel) is not as large as in the case of TiO₂ (n_{D} 2.65), work has to be performed far above a critical pigment-volume concentration, so that an additional pigment-air interface results, which then contributes decisively to the scattering and the opacity. Due to the high refractive index difference between air (n_{D} 1.0) and pigment, the opacity gain is very high and the opacity increases strongly.

“Fresnel's” formula represents a calculation of the reflection of white light in the case of perpendicular incidence, the so-called total reflection. It quantitatively describes the reflection and transmission of a flat, electromagnetic wave at a flat interface:

$$R = {r}_{s}^{2} = {( - {r}_{p} )}^{2} = {( \frac{{n}_{1} - {n}_{2}}{{n}_{1} + {n}_{2}} )}^{2} \qquad ( 1 )$$

n₁=refractive index of the color pigment, n₂=refractive index of the matrix.

When calculating the reflection of titanium dioxide in various uses according to the above-specified formula (1), and when comparing it to the reflection values in identical uses of calcium sulfate (see Table 1), it can be seen that a dosage, which is significantly higher, depending on the application medium, for example 100-times higher, or also a dosage, which is more than 400-times higher, of calcium sulfate has to be used in order to be able to attain an identical reflection as in the case of titanium dioxide.

TABLE 1
Refraction of the reflection

| | | | | | | Ratio of the |
|---|---|---|---|---|---|---|
| | | | | | | reflection, according |
| | | | | | | to the dosage ratio |
| | | n_{D} | | n_{D} | | with identical |
| Foods | n_{D} | TiO₂ | Reflection | CaSO₄ | Reflection | reflection (1:x) |
| Hard caramel | 1.53 | 2.65 | 7.2% | 1.57 | 0.02% | 431.2 |
| Jelly | 1.42 | 2.65 | 9.1% | 1.57 | 0.25% | 36.3 |
| Chocolate button | 1 | 2.65 | 20.4% | 1.58 | 4.92% | 4.2 |

Even though magnesium as well as calcium are essential for the human body and have to be consumed daily in sufficient quantities, side effects, such as, for example, diarrhea, can occur in response to an overdosage of magnesium or calcium in the form of sulfate or carbonate salts. The optimization of pigment quantities containing calcium sulfate, magnesium sulfate, or magnesium carbonate, in particular calcium sulfate, is thus particularly important.

It is thus a further object of the invention to attain an efficiency (in opacity and reflection), which comes as close as possible to the TiO₂ efficiency, with lower quantities of pigment according to the invention, if possible, per gram of a product.

Surprisingly, it has been shown that the pigment according to the invention, in particular calcium sulfate, does not have to be used in the dosages calculated by means of Fresnel's formula (1) in selected uses, when an optimal particle size of the white pigment is required.

In an advantageous further development of the invention, the mean particle size of the at least one pigment (d[4,3]) for the dye according to the invention is approx. 0.5 to 50 μm, preferably approx. 0.8 to approx. 10 μm, particularly preferred approx. 1.0 to approx. 5.0 μm. In the context of the present invention, the volume-weighted particle size, i.e. d[4,3]-sizes (the so-called De Brouckere diameter), is referred to as mean particle size of the pigment according to the invention.

The values D₅₀ and D₉₇ are used to characterize a particle size distribution of the pigment according to the invention. The value Dₓ specifies, which volume-weighted percentage x [%] of the particles lies below this particle size. For example, D₉₇ means that 97% of all particles are smaller than the specified value. D₅₀ thereby specifies the mean particle size, i.e. 50% of the particles are smaller than the specified value. Dₘₐₓ is measure for the larger particles in the sample.

The particle size distributions were measured by means of laser light diffraction using a device of the Malvern Masersizer 3000 type using the detection and calculation method according to Mie, in particular with a refractive index for calcium sulfate particles of 1.572 for calcium sulfate anhydrite. The specification of the characteristic values Dₓ including Dₘₐₓ and the minimal as well as the mean particle size, are to be understood with the usual standard deviations in the case of measurements of particle size distributions by means of laser light diffraction. The person of skill in the art can determine the standard deviation by means of multiple measurements of the respective dye or pigment, respectively.

The above-described pigment has a standard particle size distribution, D₅₀ is thereby maximally approx. 5.00 μm, preferably approx. 4 μm to approx. 1 μm; D₉₇ is maximally approx. 20 μm, preferably approx. 18 to approx. 3 μm, whereby Dₘₐₓ is approx. 30 μm to approx. 20 μm. The smallest particle has the size>100 nm. Here and hereinafter, a pigment, which was not subjected to a systematic comminution, is identified with the expression “standard particle size distribution”.

The so-called “standard” particle size distribution can in particular have the following parameters: D₅₀=3.75 μm, D₉₇=17.00 μm, Dₘₐₓ=27.20 μmm, whereby the smallest particle is larger than 100 nm, and the mean particle size of the pigment (d[4,3]) has a value of 5 μm.

In a preferred embodiment of the invention, the at least one pigment for the dye according to the invention has the following particle size distribution: D₅₀ in the range of greater than or equal to approx. 0.5 μm to less than or equal to approx. 5 μm, preferably greater than or equal to approx. 0.8 μm to less than or equal to approx. 1.7 μm, in particular D₅₀=1.15 μm or D₅₀=1.64 μm, D₉₇ in the range of greater than or equal to approx. 2 μm to less than or equal to approx. 15 μm, preferably greater than or equal to approx. 3 μm to less than or equal to approx. 8 μm, in particular D₉₇=4.00 μm, Dₘₐₓ in the range of greater than or equal to approx. 15 μm to less than or equal to approx. 30 μm, preferably greater than or equal to approx. 18 μm to less than or equal to approx. 25 μm, in Dₘₐₓ=21.20 μm, and the smallest particle is >100 nm, whereby the mean particle size of the pigment (d[4,3]) is in the range of greater than or equal to approx. 0.8 μm to less than or equal to approx. 8 μm, preferably greater than or equal to approx. 1 μm to less than or equal to approx. 2.5 μm, in particular approx. 1.5 μm or 1.95 μm.

A pigment with a “preferred particle size distribution” according to the invention can be provided, for example, by means of grinding. A pigment with a coarser, in particular “standard” particle size distribution, as described above, for instance, can in particular be produced by means of grinding, for example in a jet mill, steam jet mill, or a ball mill. The comminution can optionally be carried out as cryogenic grinding, whereby the grinding material is cooled, for example by means of liquid nitrogen or dry ice, in such a way that it becomes brittle. The strength of the material is thus reduced and the crushing properties for the comminution are improved.

A pigment with a particle size distribution, as it is specified below with regard to FIG. 5, is particularly preferred.

A suspension of the dye according to the invention in an aqueous solution of 7% by weight of sugar at pH 3, in particular set by means of citric acid, shows a turbidity, which is twice to five-times higher, in particular 2.6 to 4.9-times higher, than a pigment mixture of 95% by weight of tricalcium phosphate and 5% by weight of calcium carbonate or of 85% by weight of tricalcium phosphate and 15% by weight of calcium carbonate, and thus makes it possible to ensure a sufficiently strong coloring even under acidic conditions, as in many beverages.

The turbidity of a suspension of a dye according to the invention in an aqueous solution of 7% by weight of sugar at pH 3, in particular set by means of citric acid, additionally decreases over a period of time of 40 minutes only by 2% to 5%, preferably by 3% to 4.3%, calculated as difference of the turbidity at 40 minutes and at 0 minutes, based on the initial value, so that a stable coloration can be attained with the help of the invention even over longer periods of time even under acidic conditions.

A further object of the invention is the use of an above-described dye for whitening or—if the dye contains at least one colorant—for coloring foods, nutritional supplements, cosmetic or pharmaceutical products, preferably for whitening or coloring foods, particularly preferably for whitening or coloring confectionery or beverages. Magnesium or calcium can be used as alkaline earth metal, whereby calcium carbonate is excluded. Calcium sulfate, magnesium sulfate, and magnesium carbonate are preferred, calcium sulfate is particularly preferred, calcium sulfate anhydrite is most preferred. Anhydrite III, anhydrite IIs/IIu, or anhydrite I can be used as calcium sulfate anhydrite.

In preferred embodiment of the invention, the pigment for the dye, in particular the sulfate, carbonate, or phosphate of an alkaline earth metal has the mean particle size (d[4,3]) from approx. 0.5 to approx. 50 μm, preferably of approx. 0.8 to approx. 10 μm, particularly preferably of 1.0 bis 5.0 μm.

The invention furthermore provides a product of the food, pharmaceutical, or cosmetics industry, which contains an above-described dye or pigment, respectively. The pigment quantity in the product of the food, pharmaceutical, or cosmetics industry is at least approx. 0.1% by weight, preferably from approx. 0.1 to approx. 5% by weight, based on the total weight of the product.

A further object of the invention is the use of a sulfate, carbonate, or phosphate of at least one alkaline earth metal as carrier for soluble colorants for coloring foods, nutritional supplements, cosmetic or pharmaceutical products. Magnesium or calcium can be used as alkaline earth metal, whereby calcium carbonate is excluded. Calcium sulfate, magnesium sulfate, and magnesium carbonate are preferred, calcium sulfate is particularly preferred, calcium sulfate anhydrite is most preferred. Anhydrite III, anhydrite IIₛ/IIᵤ, or anhydrite I can be used as calcium sulfate anhydrite.

In a preferred embodiment of the invention, the sulfate, carbonate, or phosphate of an alkaline earth metal has the mean particle size (d[4,3]) from approx. 0.5 to approx. 50 μm, preferably from approx. 0.8 to approx. 10 μm, particularly preferably from 1.0 to 5.0 μm.

The invention also relates to all combinations of preferred embodiments, provided that they are not mutually exclusive. The specifications “approximately” or “approx.” in combination with a figure means that values, which are higher or lower at least by 10%, or values, which are higher or lower by 5%, and values, which are higher or lower at least by 1%, are included.

### BRIEF DESCRIPTION OF THE DRAWINGS

The invention will be described in more detail below with reference to the enclosed figures and on the basis of exemplary embodiments. The features of the different exemplary embodiments can be combined with one another in varying combination, in which:

FIG. 1 shows photographs of gelatin-based fruit gummies with pigments according to the invention according to a first and second embodiment of the invention as well as for comparison with titanium dioxide and resistant maize starch on a black background,

FIG. 2 shows photographs of hard caramels with white color according to a third and fourth embodiment of the invention compared with white color with titanium dioxide,

FIG. 3 shows photographs of chocolate-covered hazelnuts, which are sugar-coated with white color according to a fifth and sixth embodiment of the invention, compared to white color with titanium dioxide,

FIG. 4 shows a comparative illustration of the optical effect of individual sugar-coating layers on the basis of a chocolate-covered hazelnut, which is sugar-coated with a 5% sugar-coating solution with calcium sulfate particles in 20 layers, with the sugar coating of a chocolate-covered hazelnut with a 1% sugar-coating solution with titanium dioxide,

FIG. 5 shows a volume density distribution of a pigment according to a preferred embodiment of the invention, and

FIG. 6 shows illustrations of samples of two dyes according to the invention and of two pigment mixtures of tricalcium phosphate and calcium carbonate with turbidity measuring values measured directly after the production as well as 10, 20, and 40 minutes thereafter.

### DETAILED DESCRIPTION

### Exemplary Embodiments

The “%” specifications mentioned in the exemplary embodiments represent percentages by weight (wt.-%), whereby all wt.-% are based on the total weight of a corresponding composition. The proportions of the respectively mentioned components of the recipes add up to 100% by weight in each example.

### Example 1: Coloring of Gelatin-Based Fruit Gummies

The following components were used for the recipe:

44.0% by weight of glucose syrup,

32.0% by weight of sucrose,

7.0% by weight of gelatin,

1.0% by weight of citric acid, and

water

Weigh gelatin in a heat-resistant bowl and dissolve it with hot water (ratio 2:1). Provide in a heating cabinet (approx. 60° C.) until use.

Dissolve sugar (sucrose and glucose syrup) in little water, and boil in a pot to approx. 120° C. Cool down sugar mass to below 100° C. and slowly stir in dissolved gelatin. Slowly stir in pigment (e.g. calcium sulfate, titanium dioxide, or maize starch).

Pour colored fruit gummy mass into prepared starch molds and allow to cool down for at least 24 h.

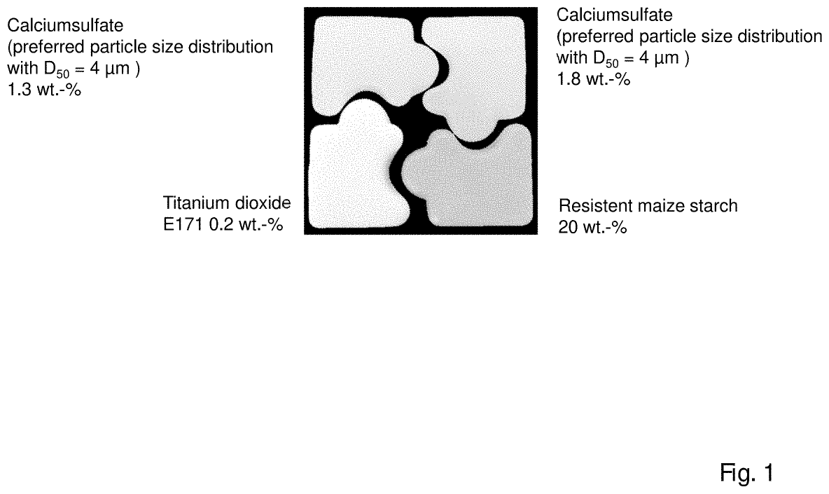

A gelatin-based fruit gummy is in each case shown on a black background in FIG. 1, namely in the illustration as pigment on the bottom left with 0.2% by weight of titanium dioxide (E171) and on the bottom right with 20% by weight of resistant maize starch, as well as on the top with a dye according to the invention, namely calcium sulfate with the preferred particle size distribution, that is, with D₅₀=4 □m, in a content of 1.3% by weight on the top left and 1.8% by weight on the top right. Compared to the resistant maize starch, a significantly improved whitening can be seen due to the use of the dye according to the invention, namely already at a concentration in the end product of 1.3% by weight.

### Example 2: Coloring of Foamed Pectin-Based Fruit Gummies

The following components were used for the recipe:

76% by weight of glucose syrup,

5% by weight of gelatin,

1.6% by weight of critic acid,

1% by weight of pectin,

0.01 to 2% by weight of calcium sulfate or titanium dioxide, and

water

Dissolve gelatin in water (ratio 2:1). Mix pectin and sugar (glucose syrup), dissolve in little water, and boil to approx. 120° C. Cool down sugar/pectin mass and slowly stir in dissolved gelatin. Whip at high speed in a suitable mixer (for example Hobart, universal food processor). Slowly stir in color pigment (e.g. calcium sulfate or titanium dioxide) and whip again at high speed (Hobart—speed 3). Deposit the mass in prepared starch molds by means of a piping bag.

### Example 3: Coloring of Hard Caramels

The following components are used for the recipe:

90 to 92% by weight of isomalt

6% by weight of maltitol

1.7% by weight of citric acid,

0.01 to 2% by weight of calcium sulfate or titanium dioxide, and

water

Boil isomalt, maltitol, and in little water (>170° C.). Allow temperature to cool down to 140° C. and add citric acid. Allow temperature to cool down to 110° C. and add pigment (e.g. calcium sulfate or titanium dioxide). Pour hot caramel mass into molds and allow to cool down.

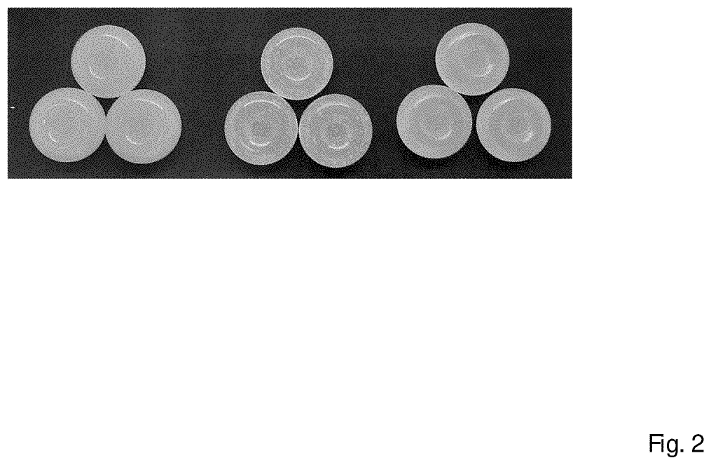

Photographs of hard caramels with white color are shown in FIG. 2. From left to right, three hard caramels can be seen, colored with 0.01% by weight of titanium dioxide, three hard caramels colored with 1.00% by weight of calcium sulfate in the preferred particle size distribution, and three hard caramels colored with 2.00% by weight of calcium sulfate in the preferred particle size distribution with D₅₀=1.15 μm, D₉₇=4.00 μm, Dₘₐₓ=21.20 μm, and the smallest particle is >100 nm, whereby the mean particle size of the pigment (d[4,3]) is 1.5 μm. A whitening, which is very close to the result of titanium dioxide as pigment for the use in hard caramels, is attained by means of the dosage of 2% by weight of the dye according to the invention with calcium sulfate as pigment.

### Example 4: Hard Sugar Coating of Chocolate Drops, Use of the Pigment in the Sugar-Coating Solution

The following components were used for the recipe of the sugar-coating solution:

68% by weight of sugar,

25 to 30% by weight of water,

2% by weight of maltodextrin, and

up to 5% by weight of calcium sulfate or 1% by weight of titanium dioxide.

Production of the sugar-coating solution in a pot: Dissolve sugar and maltodextrin in water, and boil (106° C.) until everything is dissolved. Add pigment at 35° C. by stirring. Introduce chocolate drops into coating drum. Build up several layers by alternating powdered sugar and sugar-coating solution and allow to try. Finally seal with brightening agent.

### Example 5: Sugar Coating of Chocolate-Covered Hazelnuts with White Color

The following components were used for the recipe:

70 g of sugar,

30 g of water, and

1% by weight of titanium dioxide or up to 10% by weight of calcium sulfate.

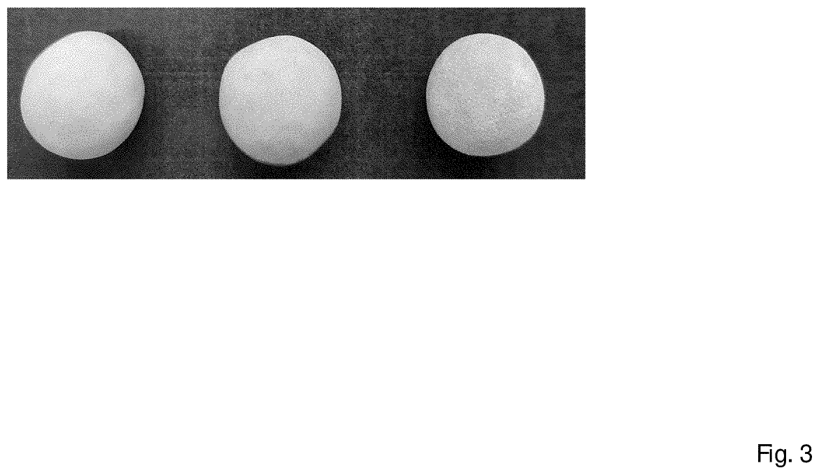

For the coating of the chocolate-covered hazelnuts according to this example 5, the same sugar-coating solutions as for the nuts shown in FIG. 3 were used, that is, the pigment CaSO₄ was present in the form of particles with the “preferred particle (size) distribution” with D₅₀=4 μm. A coating, which is comparable to the whitening power of titanium dioxide, was attained with the content of 10% by weight of calcium sulfate as pigment.

From left to right in FIG. 3, photographs of chocolate-covered hazelnuts are shown, which were coated with sugar-coating solutions according to the above-described recipe, whereby a content of 5% by weight was set for calcium sulfate. In the picture on the left, a chocolate-covered hazelnut can be seen, which is provided with 14 layers of a 1% sugar-coating solution with titanium dioxide as pigment, the particle size distribution of which has a value of D₅₀=0.25 μm.

The picture in the middle shows a chocolate-covered hazelnut, which is coated with 20 layers of a 5% sugar-coating solution with calcium sulfate as pigment according to a fifth embodiment of the invention, whereby the pigment is present in the form of particles with the particularly preferred particle size distribution, in the case of which D₅₀=1.6 μm.

In the picture on the right, a chocolate-covered hazelnut is shown, which is covered with 20 layers of a 5% sugar-coating solution with calcium sulfate as pigment according to a sixth embodiment of the invention, whereby the pigment is present in the form of particles with the preferred particle size distribution, in the case of which D₅₀=4 μm. It can be seen that when using the 5% sugar-coating solution according to the invention with calcium sulfate as pigment, a whitening of the chocolate-covered hazelnut is attained in spite of the coarser particles, which is comparable to that of the titanium dioxide-containing sugar-coating solution.

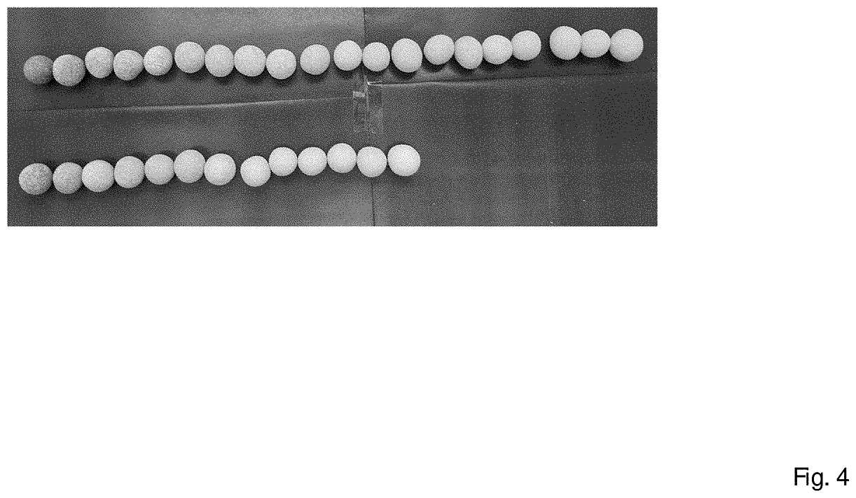

In connection with the image shown in the right picture in FIG. 3 of a chocolate-covered hazelnut, which is sugar-coated in 20 layers with a 5% sugar-coating solution with calcium sulfate particles according to the sixth embodiment of the invention described here, a comparison of the optical effect, attained when applying one of these layers each compared to a sugar coating of a chocolate-covered hazelnut with a 1% sugar coating with titanium dioxide as pigment is shown in FIG. 4. A chocolate-covered hazelnut is in each case shown in the bottom row after the application of a first pigment layer (left) of a 1% sugar-coating solution with titanium dioxide as pigment, a second layer of this sugar-coating solution (on the right next to the first image), and so on, to a hazelnut after the application of the thirteenth layer of the titanium dioxide-containing sugar-coating solution (on the very right in the top row).

A chocolate-covered hazelnut is in each case shown in the top row in FIG. 4 after the application of a first pigment layer of the 5% sugar-coating solution with calcium sulfate particles (left), a second pigment layer (on the right next to the first image), and so on, to a hazelnut after the application of the twentieth layer (on the very right in the top row). In comparison, it can be seen that a significant increase of the whitening of the chocolate-covered hazelnut is no longer attained purely optically after approximately the eleventh layer. The person of skill in the art can determine the number of layers, which are required for the respective use case with the used dyes in order to attain the desired optical effect, by means of test or reference series of this type.

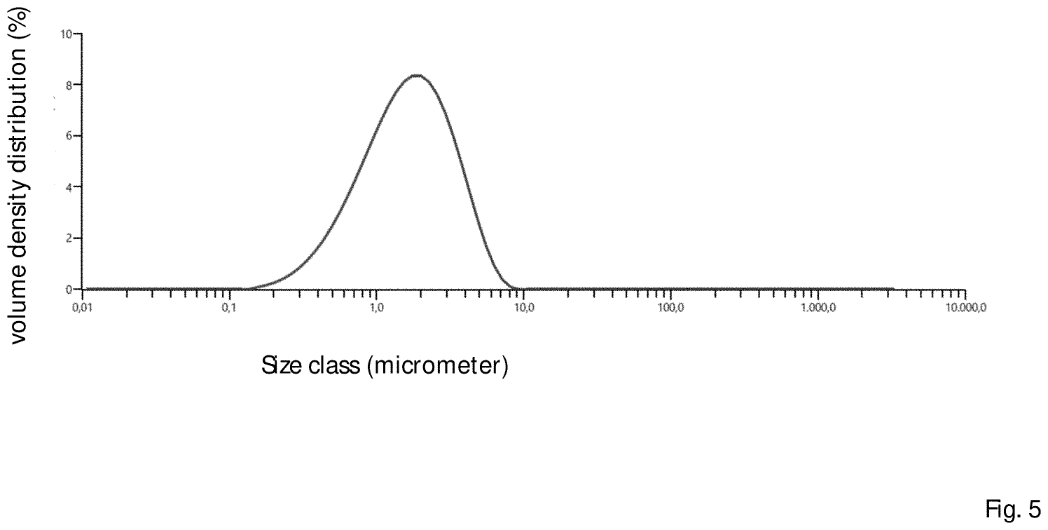

A particle size distribution for a pigment, which is particularly preferred in the context of the invention, is illustrated in FIG. 5. The pigment consists of calcium sulfate anhydrite. The analysis of the data was carried out according to the Mie model for a refractive index of the particles of 1.580 and an absorption index of the particles of 0.010 with a refraction index of the dispersing agent of 1.0 by means of a device of the model Malvern Mastersizer 3000.

The particle size analysis provided a value for the uniformity of the totality of the particles of 0.599 and a specific surface of 5045 m²/kg. The Sauter mean diameter d[3,2] is 1.19 □m. The De Brouckere diameter d[4,3] is 1.95 □m. D₁₀ is 0.593 □m, D₅₀ has a value of 1.64 □m, and D₉₀ is 3.78 □m, The smallest detected particles are larger than 128 nm and the largest detected particles are smaller than Dₘₐₓ=7.64 □m.

The particles measured with the result as illustrated in FIG. 5 and as described above, were produced by grinding a calcium sulfate anhydrite pigment, the particle size distribution of which was analyzed as follows: The Sauter mean diameter d[3,2] was 2.12 □m. The De Brouckere diameter d[4,3] was 3.27 □m. D₁₀ was 1.04 □m, D₅₀ had a value of 2.76 □m, and D₉₀ was 6.27 □m. The value for D₉₇ was 8.36 □m. The smallest detected particles were larger than 461 nm, and the largest detected particles were smaller than Dₘₐₓ=11.2 □m.

A dye with a pigment for whitening consisting of calcium sulfate anhydrite, which has the result of the particle size distribution as illustrated in FIG. 5 and as described above, was used as dye and was analyzed with regard to its pH stability, compared to a mixture of tricalcium phosphate and calcium carbonate (hereinafter “TCP” or “CC”, respectively).

A first turbidity measurement had the following result, whereby all pigments had a comparable particle size distribution. 2.75 g/L of the pigment or of the pigment mixture were in each case dispersed into a base recipe of demineralized water with 7% by weight of sugar and a quantity of citric acid.

| Pigment or pigment mixture | Turbidity/FNU |
|---|---|
| Calcium sulfate anhydrite according to the invention | 249 |
| 95% by weight of TCP + 5% by weight of CaCO3 | 190 |
| 85% by weight of TCP and 15% by weight of CaCO3) | 208 |

The turbidity was used as measure for the opacity of the samples. The turbidity was optically determined in accordance with ISO 7027 in the above-reproduced first analysis at a wavelength of the used light 860 nm under scattered light measurement at an angle of 90°, whereby the result is specified in “FNU”, that is, “Formazine Nephelometric Units”.

Calcium sulfate anhydrite according to the invention results in the highest turbidity. The second highest turbidity is attained by the 85/15 mixture (TCP/CC). This mixture, however, is not sufficiently stable due to the content of calcium carbonate, and the calcium carbonate dissolves after a relatively short time under the acidic conditions, and only the insoluble TCP remains.

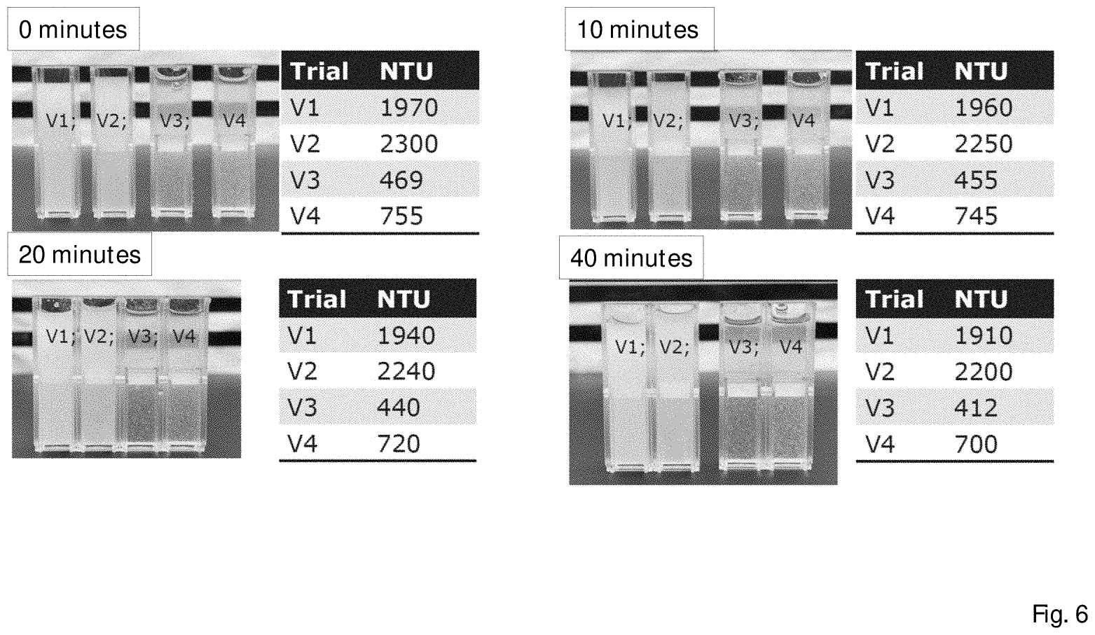

Two variations of the invention compared with the mixtures of TCP and CC were observed in a closer analysis. A sample (“trial”) of a pigment according to the invention consisting of calcium sulfate anhydrite with a particle size D₅₀ of 1.6 □m or 4 □m, respectively, will hereinafter be identified with “V1” or “V2”, respectively. A sample (“trial”) of a mixture of 85% by weight of TCP+15% by weight of CaCO3 or 95% by weight of TCP+5% by weight of CaCO3, respectively, is identified with “V3” or “V4”, respectively. To set a pH value of 3, 2.75 g/L of the pigment or of the pigment mixture were in each case dispersed in a base recipe of demineralized water with 7% by weight of sugar and a quantity of citric acid. The results are compiled in FIG. 6. The turbidity data is specified in the unit “NTU”. This “Nephelometric Turbidity Unit” (NTU) is a unit used in the water treatment for the turbidity of liquids, which is determined by means of a calibrated nephelometer.

FIG. 6 shows images of the samples in jars in front of a dark background in the bottom region and in front of black and white striped background in the top region at the beginning of the measurement (image top left in FIG. 6, time specification “0 minutes”) as well as after 10, 20, and 40 minutes. At the beginning of the measurement (“0 minutes”), a 2.6- to 4.9-times higher turbidity of the pigment according to the invention appears in the beverage base recipe than in the case of the pigment mixtures of TCP and CC.

Over a period of time of 40 minutes, the turbidity of the pigment according to the invention decreases only by 3% to 4.3% (calculated as differences of the turbidity at 40 minutes and at 0 minutes based on the initial value) in the beverage base recipe. In contrast, the turbidity of the pigment mixtures of TCP and CC in the beverage base recipe decreases over a period of 40 minutes by 7.3% to 12.1% and thus by more than double or almost three times, respectively, compared to the pigment according to the invention.

This data confirms the first measurements according to the above table and show that the pigment according to the invention effects a stable whitening of a beverage even in the case of a low pH value of 3, while the turbidity decreases significantly over time in the case of known pigment mixtures. It can furthermore be seen that the coverage of the black and white striped background remains unchanged over the entire measuring direction when using the dyes according to the invention.

The person of skill in the art can see that the invention is not limited to the above-described examples, but on the contrary, can be varied in a variety of ways. The features of the individually illustrated examples can in particular also be combined with one another or can be exchanged for each other.

1.-11. (canceled)

12. A dye for coloring foods, nutritional supplements, cosmetic or pharmaceutical products, comprising:
at least one pigment in the form of a water-insoluble sulfate, carbonate or phosphate of at least one alkaline earth metal, which is selected from the group consisting of
magnesium phosphate,
calcium phosphate,
magnesium carbonate, and
calcium sulfate anhydrite having a mean particle size (d[4,3]) from 1.0 to 5.0 μm.

13. The dye according to claim 12,
wherein a suspension of the dye in an aqueous solution of 7% by weight of sugar at pH 3 has a turbidity which is twice to five-times higher than a pigment mixture of 95% by weight of tricalcium phosphate and 5% by weight of calcium carbonate or of 85% by weight of tricalcium phosphate and 15% by weight of calcium carbonate.

14. The dye according to claim 12,
wherein a turbidity of a suspension of the dye in an aqueous solution of 7% by weight of sugar at pH 3 decreases over a period of time of 40 minutes by 2% to 5% calculated as difference of the turbidity at 40 minutes and at 0 minutes, based on an initial value.

15. The dye according to claim 12,
further comprising at least one soluble colorant.

16. A method for whitening foods, nutritional supplements, cosmetic or pharmaceutical products, comprising:
mixing a foodstuff, nutritional supplement, cosmetic product or pharmaceutical product with a dye comprising
at least one pigment in the form of a water-insoluble sulfate, carbonate, or phosphate of at least one alkaline earth metal, which is selected from the group consisting of
magnesium phosphate,
calcium phosphate,
magnesium carbonate, and
calcium sulfate anhydrite having a mean particle size (d[4,3]) from 1.0 to 5.0 μm.

17. A method for coloring foods, nutritional supplements, cosmetic or pharmaceutical products, comprising:
mixing a foodstuff, nutritional supplement, cosmetic product or pharmaceutical product with a dye comprising
at least one pigment in the form of a water-insoluble sulfate, carbonate, or phosphate of at least one alkaline earth metal, which is selected from the group consisting of
magnesium phosphate,
calcium phosphate,
magnesium carbonate, and
calcium sulfate anhydrite
with the anhydrite having a mean particle size (d[4,3]) from 1.0 to 5.0 μm,
wherein a suspension of the dye in an aqueous solution of 7% by weight of sugar at pH 3 has a turbidity which is 2 to 5-times higher than a pigment mixture of 95% by weight of tricalcium phosphate and 5% by weight of calcium carbonate or of 85% by weight of tricalcium phosphate and 15% by weight of calcium carbonate.

18. A product of the food, pharmaceutical, or cosmetics industry, comprising the dye according to claim 12.

19. The product according to claim 18,
wherein a quantity of the at least one pigment is between 0.1 to 5% by weight, based on a total weight of the product.
